(19) United States
(12) Patent Application Publication
(10) Pub. No.: US 2024/0145029 A1
(43) Pub. Date: May 2, 2024

(54) SYSTEM AND METHOD FOR DETERMINING AND PRIORITIZING A PLURALITY OF SECONDARY TARGET PROTEIN

(71) Applicant: Innoplexus AG, Eschborn (DE)

(72) Inventors: Om Prakash Sharma, Pune (IN); Vinay Chandil, Gwalior (IN); Amit S. Choudhari, Pune (IN)

(73) Assignee: Innoplexus AG, Eschborn (DE)

(21) Appl. No.: 17/978,305

(22) Filed: Nov. 1, 2022

(51) Int. Cl. G16B 15/30; G06N 20/00; G16C 20/50; G16C 20/60

(52) U.S. Cl. CPC G16B 15/30; G06N 20/00; G16C 20/50; G16C 20/60

(57) ABSTRACT

The present invention discloses a system and method for determination and prioritization of a plurality of secondary target protein. According to the invention, the present invention ensures the abstraction and authentication of possible scientific data for the primary target protein and introduces a statistical analysis of multiple similarity criteria to determine similar proteins in the form of the plurality of secondary target protein for an input query. Furthermore, the identified plurality of secondary target protein is analysed via matrix and multi-relational directed network analysis to prioritize and depict relationships among similarity concepts. Beneficially, the present invention ensures a high level of coverage and precision to descript protein-protein similarity.

### FIELD OF INVENTION

In general, the present invention relates to the field of bioinformatics. In particular, the present disclosure relates to a system and method for determining and prioritizing a plurality of similar protein in the form of secondary target protein.

### BACKGROUND

Proteomics is an emerging discipline based on the human genome project. Generally, it relates to correlating directly the involvement of specific proteins, protein complexes, molecular functions, and their modification status in a given disease state and primarily aims to determine the presence and quantity of proteins. The identification of protein sequence in proteomics is critical as it facilitates a systematic understanding of key biological knowledge including protein structure, function, and evolutionary relationship. Mass-spectrometry-based proteomics is generally the most used approach for identifying proteins and determining protein expression under various conditions to identify post-translational modifications in response to stimuli and to characterize protein interactions. Conventionally, various strategies are involved for protein identification, but the main strategies employed by mass spectrometry include database searching, de novo sequencing, and peptide sequence tag. Among these strategies, database searching is the most popular. In this approach, experimental protein spectra are compared with theoretical spectra from the database to identify the best fit.

However, numerous challenges in this field continue to persist. While protein data analysis has been greatly assisted by many bioinformatics tools developed in recent years, a careful analysis of the major steps and flow of data in a typical high throughput analysis reveals a few gaps that have not been filled by existing research solutions and need to be filled to fully realize the value of the proteomics data. Furthermore, enrichment of protein targets represents the process of identification of similar proteins and associated data therewith. The enrichment of protein targets has an important role in discovering the unknown links between drugs and disease, therefore it is crucial to identify proteins with a good amount of accuracy, in order to provide the functioning of drug discovery and repositioning. However, the identification of indirect relevant relations in proteins has always been a challenge at each step of drug discovery and repositioning. Furthermore, there are multiple scientific approaches for protein identification, and balancing between all the scientific approaches is challenging and is not possible with any of the existing research solutions.

Therefore, in light of the foregoing discussion, there exists a need to overcome the aforementioned drawbacks so as to provide a system for identifying similar proteins.

### SUMMARY

An object of the present invention is to provide a system and method for integrating numerous and diverse data sources for data processing to identify similar proteins.

Another object of the present disclosure is to prioritize the identified similar proteins based on the plurality of similarity criteria.

In a first aspect, embodiments of the present disclosure provide a system for prioritizing a plurality of secondary target protein, wherein the system comprises:
a database arrangement; and
a processor communicably coupled via a data communication network to the database arrangement, wherein the processor is configured to:
   receive information associated with a primary target protein; determine the plurality of secondary target protein similar to the primary target protein based on a plurality of similarity criteria;
   perform a matrix analysis to assign weights to each of the similarity criteria from the plurality of similarity criteria; build a multi-relational directed network from the determined plurality of secondary target protein;
   perform a clustering operation on the multi-relational directed network via a clustering algorithm, to group the multi-relational directed network into a plurality of clusters;
   process the plurality of clusters to identify one or more relevant clusters, wherein the one or more relevant clusters include the primary target protein and one or more relevant secondary target protein connected directly and/or indirectly with the primary target protein; and
   determine a priority sequence of the one or more relevant secondary target protein connected to the primary target protein based on the assigned weights to each of the similarity criteria.

Optionally, the processor is configured to validate the received information associated with the primary target protein based on authentication and abstraction of data using one or more ontologies, wherein the data relates to the information associated with the primary target protein.

Optionally, the one or more ontologies correspond to at least a protein ontology and a gene ontology.

Optionally, the set of similarity criteria comprises at least one of protein-protein interaction, molecular function similarity, protein sequence similarity, and disease target similarity.

Optionally, the data that describes the primary target protein comprises at least sequence information, function classification information, metabolic pathway information, interaction profile, and Gene Ontology functional annotation of the primary target protein.

Optionally, the protein-protein interaction is based on closeness centrality of the primary target protein with respect to the plurality of secondary target protein.

Optionally, the processor is configured to assign weights to each of the similarity criteria based on an analysis of multi-criteria decision-making matrix.

Optionally, the processor is configured to calculate priority scores associated with each of the similarity criteria for calculating the weights to be assigned to each of the similarity criteria, wherein the priority scores determine the importance of a similarity criteria with respect to other similarity criteria.

Optionally, the multi-relational directed network defines a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

Optionally, the processor is configured to calculate direct scores and/or indirect scores of the relevant secondary target protein based on the weights of the similarity criteria assigned to each of the edges defined in the multi-relational directed network.

In a second aspect, embodiments of the present disclosure provides a computer-implemented method for prioritizing a plurality of secondary target protein, wherein the method comprises:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

Optionally, the method comprises validating the received information associated with the primary target protein based on authentication and abstraction of data using one or more ontologies, wherein the data relates to the information associated with the primary target protein.

Optionally, the one or more ontologies correspond to at least a protein ontology and a gene ontology.

Optionally, the set of similarity criteria comprises at least one of protein-protein interaction, molecular function similarity, protein sequence similarity, and disease target similarity.

Optionally, the data that describes the primary target protein comprises at least sequence information, function classification information, metabolic pathway information, interaction profile, and Gene Ontology functional annotation of the primary target protein.

Optionally, the protein-protein interaction is based on closeness centrality of the primary target protein with respect to the plurality of secondary target protein.

Optionally, the method comprises assigning weights to each of the similarity criteria based on an analysis of multi-criteria decision-making matrix.

Optionally, the method comprises calculating priority scores associated with each of the similarity criteria for calculating the weights to be assigned to each of the similarity criteria, wherein the priority scores determine the importance of a similarity criteria with respect to other similarity criteria.

Optionally, the method comprises defining a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

Optionally, the method comprises calculating direct scores and/or indirect scores of the relevant secondary target protein based on the weights of the similarity criteria assigned to each of the edges defined in the multi-relational directed network.

In a third aspect, embodiments of the present disclosure provide a non-transitory computer readable storage medium, containing program instructions for execution on a computer system, which when executed by a computer, cause the computer to perform method steps of a method for prioritizing a plurality of secondary target protein, the method comprising the steps of:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

A better understanding of the present invention may be obtained through the following examples which are set forth to illustrate but are not to be construed as limiting the present invention.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a block diagram of a system for prioritizing a plurality of secondary target protein, in accordance with an embodiment of the present disclosure;

FIG. 2 is a schematic illustration of a flow diagram of the system for determining and prioritizing the plurality of secondary target protein, in accordance with an embodiment of the present disclosure;

FIG. 3 is a schematic illustration for the determination of similar proteins, in accordance with another embodiment of the present disclosure; and

FIG. 4 is a schematic illustration of the steps of a method for prioritizing a plurality of secondary target protein, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In a first aspect, embodiments of the present disclosure provide a system for prioritizing a plurality of secondary target protein, wherein the system comprises:
a database arrangement; and
a processor communicably coupled via a data communication network to the database arrangement, wherein the processor is configured to:
   receive information associated with a primary target protein;
   determine the plurality of secondary target protein similar to the primary target protein based on a plurality of similarity criteria;
   perform a matrix analysis to assign weights to each of the similarity criteria from the plurality of similarity criteria; build a multi-relational directed network from the determined plurality of secondary target protein;
   perform a clustering operation on the multi-relational directed network via a clustering algorithm, to group the multi-relational directed network into a plurality of clusters;
   process the plurality of clusters to identify one or more relevant clusters, wherein the one or more relevant clusters include the primary target protein and one or more relevant secondary target protein connected directly and/or indirectly with the primary target protein; and
   determine a priority sequence of the one or more relevant secondary target protein connected to the primary target protein based on the assigned weights to each of the similarity criteria.

In a second aspect, embodiments of the present disclosure provides a computer-implemented method for prioritizing a plurality of secondary target protein, wherein the method comprises:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

The present disclosure provides the aforementioned system and a method for integrating numerous and diverse data sources for data processing to identify similar proteins and prioritizing them thereafter. More specifically, embodiments of the present invention are related to one or more algorithms employed to predict the similar protein hypotheses based on multiple scientific aspects and/or parameters and/or criteria of protein sequence similarity, disease target similarity, interacting proteins and molecular function similarity.

Embodiments of the present invention permit the identification of proteins similar to the proteins that have been queried in a database arrangement. Furthermore, the present disclosure relates to the determination of the new indications for an existing drug. However, it requires a wide range of biological information, particularly the information associated with the proteins. In principle, a drug targeting protein interacts with other proteins to regulate signalling pathways, molecular function and activities within a biological system. Conventionally, the identification of indirect relevant relations has always been a challenge at each step of drug discovery and repositioning. Enrichment of protein targets has important role in discovering the unknown links between drugs and disease. The present invention hereby facilitates the complete coverage of similar proteins based on multiple scientific rationale resulting in outcome with increased accuracy for pathway and disease enrichment. In an embodiment, the multiple scientific rationale is selected from the group, but not limited to protein-protein interaction similarity, molecular function similarity, protein sequence similarity and disease target similarity and so forth. In a preferrable embodiment, the multiple scientific rationale includes the combination of protein-protein interaction similarity, molecular function similarity, protein sequence similarity and disease target similarity.

Beneficially, such analysis is employed to identify a large data set of protein targets, wherein one or more structural and functional integrity of the identified proteins may be exploited for targeted drug development. Advantageously, the present invention facilitates the drug discoverers to propose therapeutics for rare and incurable diseases.

Throughout the present disclosure, the term “database arrangement” as used herein, relates to an organized body of digital information regardless of the manner in which the data or the organized body thereof is represented. It refers to a collection of data that allows easy access, management, and updating of the data stored. Optionally, the database arrangement may be hardware, software, firmware, and/or any combination thereof. For example, the organized body of digital information may be in a form of a table, a map, a grid, a packet, a datagram, a file, a document, a list or in any other form. Optionally, the data in the database arrangement is organized into rows, columns, and tables. Additionally, optionally, the data in the database arrangement is indexed (namely, labelled) for easy access thereto. Optionally, the database arrangement comprises a set of processes (namely instructions) to create the plurality of databases and update thereto, query data from external sources, and process operational instructions provided thereto. Optionally, the database arrangement is accessed electronically for, for example storing data, accessing data, and updating data, using a computing device. More optionally, such a computing device employs a database management system (DBMS) for creating and managing the database arrangement. Furthermore, optionally, the database arrangement is an object-oriented database, SQL database, relational database, distributed database, non-SQL database, cloud database. The plurality of databases includes any data storage software and systems, such as, for example, a relational database like IBM DB2®, Google Cloud and Oracle 9®. Furthermore, the database arrangement also includes a software program for creating and managing one or more databases. Optionally, the database arrangement may be operable to support relational operations, regardless of whether it enforces strict adherence to a relational model, as understood by those of ordinary skill in the art. Additionally, the database arrangement is populated by the elastic search libraries, elastic search databases, at least one relevant data element, topic-based web content and the likes. Optionally, the database arrangement is populated by the operational data associated with the URIs, URLs, and/or URNs and their related information.

Throughout the present disclosure, the term “processor” as used herein relates to at least one programmable or computational entity configured to acquire process and/or respond to instructions identifying related proteins. For example, the computational entity may include a memory, a network adapter and the likes. In another example, the processor includes, but are not limited to, a microprocessor, a microcontroller, a complex instruction set computing (CISC) microprocessor, a reduced instruction set (RISC) microprocessor, a very long instruction word (VLIW) microprocessor, or any other type of processing circuit for executing data management and curation instructions. Furthermore, the processor includes one or more processing devices and various elements of a computer system associated with a processing device that may be shared by other processing devices. Additionally in an embodiment, one or more processors, processing devices, processing devices, and elements are arranged in various architectures for responding to and processing the instructions that drive the system for determining related proteins.

Throughout the present disclosure, the term “data communication network” refers to individual networks or a collection thereof interconnected with each other and functioning as a single large network. Optionally, such a data communication network is implemented by way of a wired communication network, wireless communication network, or a combination thereof. It will be appreciated that a physical connection is established for implementing the wired communication network, whereas the wireless communication network is implemented using electromagnetic waves. Examples of such data communication networks include, but are not limited to, Local Area Networks (LANs), Wide Area Networks (WANs), Metropolitan Area Networks (MANs), Wireless LANs (WLANs), Wireless WANS (WWANs), Wireless MANS (WMANs), the Internet, second-generation (2G) telecommunication networks, third generation (3G) telecommunication networks, fourth-generation (4G) telecommunication networks, fifth-generation (SG) telecommunication networks and Worldwide Interoperability for Microwave Access (WiMAX) networks.

Optionally, the data communication network is implemented as a cellular network. It will be appreciated that the cellular network refers to a radio communication network, wherein the cellular network is distributed over land through cells. Specifically, each cell includes a fixed location transceiver, for example, a base station.

In general, the term “secondary target protein” refers to the one or more proteins that are similar to the proteins whose information have been queried in a database arrangement. The present invention aims on determining a plurality of similar protein in the form of secondary target protein. In a particular embodiment, the similarity between the proteins is based on similarity criteria, comprising but not limited to protein-protein interaction, molecular functional similarity, protein sequence similarity, and disease target similarity.

According to the present disclosure, the processor receives information associated with a primary target protein as input query. Optionally, the information associated with the primary target protein comprises the physical and/or chemical properties of the amino acids of proteins that further determine the biological activity of the protein. Said properties are used for the analysis of amino acids and the number of charged residues, basic residues, and acidic average molecular weight. Beneficially, they are important clues as well as the PPI topological features for the judgment of which proteins could be the targets. Additionally, they can provide information on whether a protein is suitable to be a drug target protein. Optionally, the information associated with the primary target protein comprises the data associated with the protein's sequence, its post-translational modifications, germline variants, expression profile and drug target status. Optionally, the information associated with the primary target protein consisting of all G-protein coupled receptors, ion channels, kinases and proteases, as well as proteins that are implicated in rare and incurable disease, for example cancer.

Optionally, the primary target protein is selected and/or received as input, consisting of specific functionalities such as structural, functional categories, physiological role, gene type, EC scheme, taxonomy of genes, taxonomy of pathways, taxonomy of reactions, taxonomy of ligand/compound, subcellular localization, protein classes, protein complexes, phenotypes, pathways, genetic element type, cellular role, molecular environment, genetic properties, post translational modifications, gene identification list, protein design and mutant stability and affinity prediction (EGAD), cellular roles, metabolic classification, cellular component, process, phylogenetic classification database and any combination thereof.

In accordance with an embodiment of the present disclosure, the data that describes the primary target protein structure comprises at least sequence information, function classification information, metabolic pathway information, interaction profile, and Gene Ontology functional annotation of the primary target protein structure.

Throughout the present disclosure, the term “one or more ontologies” as used herein relates to a set of concepts (namely, information, ideas, data, semantic associations, and so forth) in a field (namely, subject area, domain, and so forth) that details types and properties of the set of concepts and semantic association thereof. Furthermore, the ontology provides a base for validating one or more primary target proteins and thereby determining the related secondary target protein as desired. Moreover, ontology provides a structured, optimal, and relevant set of concepts pertaining to the user's field of interest. Furthermore, the ontology may be used in scientific research, academic studies, market analysis, and so forth. Optionally, the ontology may include concepts in form of text, image, audio, video, or any combination thereof. Additionally, the ontology may provide information on how a certain concept in a certain field may be associated with one or more concepts in multiple fields.

Optionally, one or more ontologies include the data associated with the gene and different types of protein structure. The one or more ontologies correspond to at least a protein ontology and a gene ontology. Optionally, the one or more ontologies as described herein comprises the proprietary gene and protein ontologies.

According to an embodiment of the present disclosure, the processor is configured to validate the received information associated with the one or more primary target protein based on authentication and abstraction of data using one or more ontologies, wherein the data relates to the information associated with the one or more primary target protein. Advantageously, the processing of only the essential data instead of the combination of essential data and the redundant data reduce the processing power of the system. Furthermore, it also leads to providing relevant results by using relatively a small amount of processing power, thereby making the system more efficient as compared to the existing inventions.

Optionally, the primary target protein is validated based on different approach evidence like molecular function, biological process and so forth, in one or more ontologies which connect such primary target protein directly and/or indirectly in a disease condition.

In another embodiment, the received information associated with the primary target protein is authenticated based on a set of predefined parameters stored in the database arrangement. Optionally, the set of predefined parameters include, but not limited to degree centrality, betweenness centrality, closeness centrality, and/or Burt's constraint of the proteins. Furthermore, the abstraction of the primary target protein is executed by the processor so as to reduce the protein data to a simplified representation of the whole. In the abstraction of protein data/information, the processor removes or take away the redundant characteristics of the information in order to reduce the protein information to a set of essential characteristics.

Optionally, the processor utilizes abstraction and/or hierarchical data structures, to analyse and validate the received primary target protein. Herein, the abstraction involves object-oriented program data that represents protein structure via multiple classes and methods that together may be used to generate, process, and store structural data. The supervised and/or unsupervised machine learning employed herein, manipulate coordinate-related data structures.

Optionally, the processor validates the one or more primary target proteins to determine if the primary target protein are within the range of targets of approved drugs or the range of targets of rejected drugs (safeness of targeted proteins), via at least one of degree centrality, betweenness centrality, closeness centrality, and/or Burt's constraint of the proteins. Particularly, the processor may require the centrality measures of the proteins and the list stored in the database arrangement to determine the ranges of values assuming targets of approved drugs and targets of rejected drugs.

The validated primary target protein after being processed via authentication and abstraction process, sent for further processing of determining the plurality of secondary target protein similar to the primary target protein based on a plurality of similarity criteria. The plurality of secondary target protein may be regarded as the proteins having similar characteristics with the primary target protein.

In the present disclosure, the processor is configured to determine a plurality of secondary target protein similar to the primary target protein based on the plurality of similarity criteria. In an embodiment, the processor analyze or more specifically, statistically analyze the one or more parameters associated with protein-protein interaction, molecular function, protein sequence, and disease target of the input primary target protein. Based on the analysis of one or more parameters of the primary target protein, the processor thereby generates a set of similarity criteria that may be employed for determining a plurality of secondary target protein similar to the inputted primary target protein. Herein, the similarity criteria used for determining the plurality of secondary target protein comprises at least one of, but not limited to protein-protein interaction, molecular function similarity, protein sequence similarity, and disease target similarity. In a preferable embodiment, the similarity criteria comprise the aggregation of the similarity criteria, i.e., the aggregation of protein-protein interaction, molecular function similarity, protein sequence similarity, and disease target similarity. Herein, the processor is configured to identify a plurality of secondary target protein based on said set of similarity criteria, from the database arrangement, in response to the primary target protein received as input.

Throughout the present disclosure, as in accordance with various embodiments of the present disclosure, the term “protein-protein interaction” relates to a pivotal aspect of protein function. It will be appreciated that almost every cellular process relies on transient or permanent physical binding of two or more proteins in order to accomplish the respective task. Protein-protein interaction networks (PPIN) are mathematical representations of the physical contacts between proteins in the cell. These contacts: (1) are specific; (2) occur between defined binding regions in the proteins; (3) have a particular biological meaning (i.e., they serve a specific function). The most important interaction protein to the query target is based on the between the target based on the number of molecular functions the target shares among them centrality score

In accordance with an embodiment of the present disclosure, the protein-protein interaction is based on closeness centrality of the primary target protein with respect to the plurality of secondary target protein. The closeness centrality according to the present invention depicts how close the target is to other targets in the network. It is calculated as the sum of the path lengths from the given target to all other targets. In other words, closeness centrality basically means how close this target is to other targets and hence plays an important role in the network. It is calculated as 1/(average of distance to all nodes in a network) where N is total number of nodes and d(y, x) denotes the distance between node(y) and node(x)

$$C ( x ) = \frac{N}{{\sum }_{y} d ( y , x )}$$

Throughout the present disclosure, the term “molecular function”, in accordance with the embodiments of the present disclosure, refers to the process that can be carried out by the action of a single macromolecular machine, usually via direct physical interactions with other molecular entities. Herein, the function denotes an action, or activity, that a gene product performs. These actions are so described from two distinct but related perspectives: (1) biochemical activity, and (2) role as a component in a larger system/process.

According to an embodiment, the processors is further configured to determine a molecular function similarity score based on a ratio of a number of molecular functions specific to the primary target protein structure and the plurality of secondary target protein structures to a number of all molecular functions specific to both the primary target protein structure and the plurality of secondary target protein. In another embodiment, the molecular function similarity corresponds to the determined molecular function similarity score.

Optionally, the Molecular function similarity score was calculated using the Jaccard index.
where,
A=(Molecular function target A),
B=(Molecular function target B)

$$J ( A , B ) = \frac{\vert A ⋂ B \vert}{\vert A ⋃ B \vert}$$

Throughout the present disclosure, the term “protein sequence”, in accordance with the embodiments of the present disclosure, refers to the practical process of determining the amino acid sequence of all or part of a protein. This may serve to identify the protein or characterize its post-translational modifications. Herein, the post-translational modification (PTM) refers to the covalent and generally enzymatic modification of proteins following protein biosynthesis. Large-scale protein sequence comparison is an important but compute-intensive task in molecular biology.

For comparative analysis of the protein sequences, the processor according to one of the embodiments of the present disclosure produces alignments between a query (primary target protein) and a sequence database, both of which consist of strings over a protein alphabet Σ of a number of possible residues. The quality of an alignment is judged by its score, which is computed as follows. Each pair of aligned residues (x, y) is assigned a score δ(x, y), where δ is a |Σ|×|Σ| matrix of (mostly negative) small integer scores that assigns higher scores to pairs of identical residues and to pairs of residues that are biologically similar. δ is defined by the biological end-users of the system as defined herein, either from empirical observation or from an evolutionary model of mutation. Each run of k consecutive unaligned residues in an alignment is assigned a gap penalty −go −k·ge, where go and ge are constants. The total score of an alignment is then the sum of scores for all its aligned residue pairs, plus the sum of penalties for all its gaps.

S=Σidentities,mismatches)−Σ(gap penalties)

Score=Max(S)

In an embodiment, this score is compared to a threshold to determine whether the alignment is worth reporting to a user. According to one of the embodiments as described herein, the protein sequence similarity corresponds to the similarity of a sequence of the primary target protein structure with respect to each of the plurality of secondary target protein structures.

Throughout the present disclosure, the term “disease target similarity” as used herein relates to the target disease that a protein has its effect on for the disease treatment. Based on the assumption that similar proteins with similar functions tend to be associated with similar diseases, and vice versa, the interaction profile of protein p(i) is denoted by a binary vector IP(p(i)) representing whether protein p(i) is interacted with each disease or not. Then, the kernel for the two proteins p(i) and p(j) are defined to calculate the Gaussian kernel similarity based on their interaction profiles, which are defined as follows:

$$\begin{matrix}{KM}_{1} ( p ( i ) , p ( j ) ) = exp ( - {γ}_{p} {‖ IP ( p ( i ) ) - IP ( p ( j ) ) ‖}^{2} ) \\ {γ}_{p} = {γ}^{′} / ( \frac{1}{np} {\sum }_{γ = 1}^{np} {‖ IP ( p ( i ) ) ‖}^{2} )\end{matrix}$$

where γp is calculated by normalizing γ′p which divides the average number of associated diseases for all targets. γ′p, is set to 1 again.

In accordance with one of the embodiments of the present disclosure, wherein the one or more processors are further configured to:
determine binary vectors corresponding to a first interaction profile of the primary target protein structure and a second interaction profile of a secondary target protein structure, wherein each of the first interaction profile and the second interaction profile represents an interaction of the primary target protein structure and the secondary target protein structure, respectively, with a disease;
determine a bandwidth parameter for tuning kernel bandwidth with normalizing original parameter based on an average number of associated diseases for the primary target protein structure and the plurality of secondary target protein structures, wherein the bandwidth parameter controls the kernel bandwidth;
calculate Gaussian Kernel similarity based on the first interaction profile of the primary target protein structure and the second interaction profile of the secondary target protein structure; and determine the disease target similarity based on the calculated Gaussian Kernel similarity.

According to an embodiment, the processor employs the aggregation or combination of the similarity criteria in addition to the retrieval algorithm to determine the plurality of secondary target protein, in response to the queried information associated with the primary target protein. The database arrangement as used herein are selected from a group comprising of protein data bank (PDB), the Research Collaboratory for Structural Bioinformatics (RCSB) PDB, ASTRAL, Database of Macromolecular Movements, Dynameomics, JenaLib, ModBase, OCA, KEGG: Genes, KEGG: Pathways, KEGG: Ligand/Compound, KEGG: Ligand/Enzyme, WIT, OMIM, PDB select, Pfam, PubMed, SCOP, SwissProt, OPM, PDBe, PDB Lite, PDBsum, PDBTM, PDBWiki, ProtCID, Protein, Proteopedia, ProteinLounge, SWISS-MODEL Repository, TOPSAN, UniProt, Swiss-Prot, UniProtKB/Swiss-Prot, ExPASy, PANTHER, BioLiP, STRING, ProFunc, PROTEOME database, database of Clusters of Orthologous Groups of proteins (COG), Enzyme Commission number (EC number) database, GenProtEC, EcoCyc, MIPS: MYGD, MIPS: MATD, PEDANT, Proteome.com: YDP and WormPD, MGI: Mouse Genome Database (MGD), TIGR: Microbial databases TIGR: Expressed Gene Anatomy Database, EGAD, Gene Ontology, Institute Pasteur SubtiList, Institute Pasteur TubercuList, Sanger Centre and any combination thereof.

Optionally, the processor identifies a plurality of secondary target protein similar to the one or more primary target protein. It comprises a step of generating training data for determination of similar proteins. The generation of training data comprises steps of:
obtaining one or more primary target proteins as input;
reducing redundancy of said one or more primary target protein;
obtaining a protein sequence similarity parameter corresponding to the analysis of protein sequence of the one or more primary target protein;
obtaining a target disease similarity parameter based on the analysis of the one or more primary target protein;
obtaining a protein-protein interaction similarity based on the analysis of the protein-protein interaction of the one or more primary target protein;
obtaining a molecular function similarity based on the analysis of the one or more primary target protein;
defining a threshold value for the protein sequence similarity, disease target similarity, protein-protein interaction similarity, and molecular function similarity; and
defining training data parameters for relatedness associated with the one or more similarity criteria and their threshold values via supervised and/or unsupervised machine learning algorithm.

Optionally, the processor further generates a plurality of secondary target protein based on the training data of the protein sequence similarity, disease target similarity, protein-protein interaction similarity, and molecular function similarity, in accordance with the pre-defined embodiment of the present disclosure. Conventionally, there are instances wherein it is very cumbersome for protein identification tools to tradeoff between false positives and false negatives. It is essential to keep false positives to a minimum value during protein identification because identifying the wrong protein can lead to a costly waste of time and resources. At the same time, it is desirable to identify as many proteins as possible to draw maximum benefit from the experimental data. The ability of an algorithm to identify a protein is said to be its sensitivity, and its ability to distinguish true positives from false positives is said to be its specificity. Optionally, the processor is configured to trade-off between the true positives and the false positives, by employing a threshold value of the plurality of similarity criteria, above which proteins are classed as identified or determined.

The processor further performs a matrix analysis to assign weights to each of the similarity criteria from the plurality of similarity criteria. In an embodiment, the matrix to be used for performing matrix analysis to assign weights to each of the similarity criteria is multi-criteria decision-making matrix. Optionally, the processor assigns weights to each of the similarity criteria based on the analysis of AHP in matrix.

In an embodiment, the processor is configured to calculate priority scores associated with each of the similarity criteria for calculating the weights to be assigned to each of the similarity criteria, wherein the priority scores determine the importance of a similarity criteria with respect to other similarity criteria. The analysis of multi-criteria decision-making matrix refers to determining the relative importance of similarity criteria with respect to the other similarity criteria. For example, there are four similarity criteria such as protein-protein interaction, protein sequence similarity, molecular function similarity and disease target similarity. The processor determines the priority score of molecular function similarity with respect to the protein sequence similarity to be ⅕. The priority score ⅕ denotes that the molecular functional similarity is ⅕ times important than the protein sequence similarity. On the other hand, the priority score ⅕ denotes that the protein sequence similarity is 5 times important than the molecular functional similarity. It will be appreciated that the degree of relative importance of the similarity criteria is directly proportional to the priority score of one similarity criteria with respect to the other similarity criteria.

TABLE 1

| | Protein | Molecular | Sequence | Disease |
|---|---|---|---|---|
| | interaction | function | similarity | similarity |
| Protein | 1 | 5 | 5 | 3 |
| interaction | | | | |
| Molecular | 1/5 | 1 | 5 | 3 |
| function | | | | |
| Sequence | 1/5 | 1/5 | 1 | 1/5 |
| similarity | | | | |
| Disease | 1/5 | 1/5 | 5 | 1 |
| similarity | | | | |

Optionally, the processor is configured to determine the values of the priority scores inputted therein the multi-criteria decision-making matrix based on fuzzy importance criteria range. In an embodiment, the processor employs a machine learning module to determine the value of the priority scores based on the fuzzy importance range and utilize the determined priority scores to assign weights to each of the similarity criteria. The fuzzy importance range tells how important a similarity criterion is than the other similarity criterion.

The processor is configured to calculate the weights assigned to each of the similarity criteria based on the priority scores assigned in the multi-criteria decision-making matrix. Optionally, the processor determines the normalized pair wise matrix for the multi-criteria decision-making matrix in which the values of priority scores have been filled. The elements of the normalized pair wise matrix are determined via the processor by dividing the elements of a column of the matrix to a sum of column. Furthermore, the processor is configured calculate criteria weights or weights to be assigned to each of the similarity criteria by averaging the values inside each of the row of normalized-pair wise matrix.

The processor thereafter assigns the calculated criteria weights to each of the similarity criteria. The Table 2 shows an example of criteria weights being assigned to each of the similarity criteria.

TABLE 2

| Similarity Criteria | Weight |
|---|---|
| Protein interaction | 0.55251879 |
| Molecular function | 0.240345781 |
| Sequence similarity | 0.05411873 |
| Disease similarity | 0.15301679 |

In a particular embodiment, in a case wherein one or more similarity criteria are either added or removed from the plurality of similarity criteria, the weights are translated or distributed between the rest of the similarity criteria. In an example, there are four different similarity criteria, and another similarity criteria, i.e., biological process similarity is added therein, then the assigned weights will be distributed across all the similarity criteria including the added similarity criteria. In another example, if a particular similarity criterion is removed, the weights are then distributed between rest of the similarity criteria.

The processor is further configured to build a multi-relational directed network from the determined plurality of secondary target protein. Optionally, the multi-relational directed network defines a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

In an example, a secondary target protein obtains the scores from three similarity criteria, then there will be three edges between two proteins, the processor assigns the scores and weights to each edge. In a further example, the processor build a multi relational directed network G(V, E(w)), where V is the collection of the plurality of secondary target protein and E is the set of edges between 2 proteins (input_protein & output_protein) containing the score of the approach as weights to accommodate all four approaches.

Since, there are thousands of secondary target proteins, the multi-relational directed network is formed to be a dense network, wherein one or more edges are connecting two secondary target proteins from the plurality of secondary target protein. Optionally, the edges connecting the two secondary target protein may vary from 1, 2, 3, 4 and so forth, wherein each edge represents a particular similarity criteria.

The processor is further configured to perform a clustering operation on the multi-relational directed network via a clustering algorithm, to group the multi-relational directed network into a plurality of clusters. Optionally, the processor employs clustering algorithm is selected from the group comprising at least one of, but not limited to K-means clustering algorithm, DBSCAN clustering algorithm, Gaussian Mixture Model algorithm, BIRCH algorithm, Affinity Propagation clustering algorithm, Mean-Shift clustering algorithm, OPTICS algorithm, Agglomerative Hierarchy clustering algorithm, Divisive Hierarchical clustering algorithm, Spectral Clustering, Mini-Batch K-means algorithm and so forth. It will be appreciated that the clustering algorithm divide the plurality of nodes in the multi-relational directed network acting as secondary target protein and the connecting edges, into a number of groups such that proteins in the same cluster are more similar to other protein in the same cluster and dissimilar to the protein in another cluster.

In an embodiment, the processor is configured to calculate direct scores and/or indirect scores of one secondary target protein with respect to the other secondary target protein in the plurality of clusters.

The processor thereafter processes the plurality of clusters to identify one or more relevant clusters, wherein the one or more relevant clusters include the primary target protein and one or more relevant secondary target protein connected directly and/or indirectly with the primary target protein. Optionally, the identified relevant cluster is the cluster that is directed connected to all of the plurality of similarity criteria via edges of the network and in turn connected to the primary target protein around which the network is built. The primary target protein is the protein for which the information was received by the processor as input query. In an embodiment, the processor further determines the plurality of relevant secondary target protein that are connected directly and indirectly with the identified primary target protein. Beneficially, the relevant secondary target protein connected to the primary target protein directly and/or indirectly, are the most similar to the input protein (primary target protein), irrespective of the similarity criteria and weight or scores assigned to the similarity criteria.

The processor determines a priority sequence of the one or more relevant secondary target protein connected to the primary target protein based on the assigned weights to each of the similarity criteria. Optionally, the processor is configured to calculate direct scores and/or indirect scores of the relevant secondary target protein based on the weights of the similarity criteria assigned to each of the edges defined in the multi-relational directed network. Since the identified relevant cluster is itself a small network, some of the relevant secondary target protein are connected directly to the primary target protein. The edges connecting said primary target protein and the one or more relevant secondary target protein directly have direct scores which are calculated based on the assigned weights to each of the similarity criteria. The relevant secondary target protein having greater direct score will be having greater priority than that of the protein having lesser direct score.

Furthermore, the one or more relevant secondary target protein are also connected indirectly to the primary target protein and the one or more edges connecting said primary target protein and the one or more relevant secondary target protein indirectly have indirect scores which are calculated based on the assigned weights to each of the similarity criteria. The relevant secondary target protein having greater indirect score will be having greater priority than that of the protein having lesser indirect score.

Additionally, the one or more relevant secondary target protein are also connected directly as well as indirectly to the primary target protein and the one or more edges connecting said primary target in protein and the one or more relevant secondary target protein directly and indirectly have direct score and indirect scores which are calculated based on the assigned weights to each of the similarity criteria. The relevant secondary target protein connected directly with the primary target protein will be having greater priority than that of the protein connected indirectly with the primary target protein.

The present disclosure also relates to the method as described above. Various embodiments and variants disclosed above apply mutatis mutandis to the method.

Optionally, the method comprises validating the received information associated with the primary target protein based on authentication and abstraction of data using one or more ontologies, wherein the data relates to the information associated with the primary target protein.

Optionally, the one or more ontologies correspond to at least a protein ontology and a gene ontology.

Optionally, the set of similarity criteria comprises at least one of protein-protein interaction, molecular function similarity, protein sequence similarity, and disease target similarity.

Optionally, the data that describes the primary target protein comprises at least sequence information, function classification information, metabolic pathway information, interaction profile, and Gene Ontology functional annotation of the primary target protein.

Optionally, the protein-protein interaction is based on closeness centrality of the primary target protein with respect to the plurality of secondary target protein.

Optionally, the method comprises assigning weights to each of the similarity criteria based on an analysis of multi-criteria decision-making matrix.

Optionally, the method comprises calculating priority scores associated with each of the similarity criteria for calculating the weights to be assigned to each of the similarity criteria, wherein the priority scores determine the importance of a similarity criteria with respect to other similarity criteria.

Optionally, the method comprises defining a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

Optionally, the method comprises calculating direct scores and/or indirect scores of the relevant secondary target protein based on the weights of the similarity criteria assigned to each of the edges defined in the multi-relational directed network.

The present disclosure also relates a non-transitory computer readable storage medium, containing program instructions for execution on a computer system, which when executed by a computer, cause the computer to perform method steps of a method for prioritizing a plurality of secondary target protein, the method in comprising the steps of:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

### DETAILED DESCRIPTION OF THE DRAWINGS

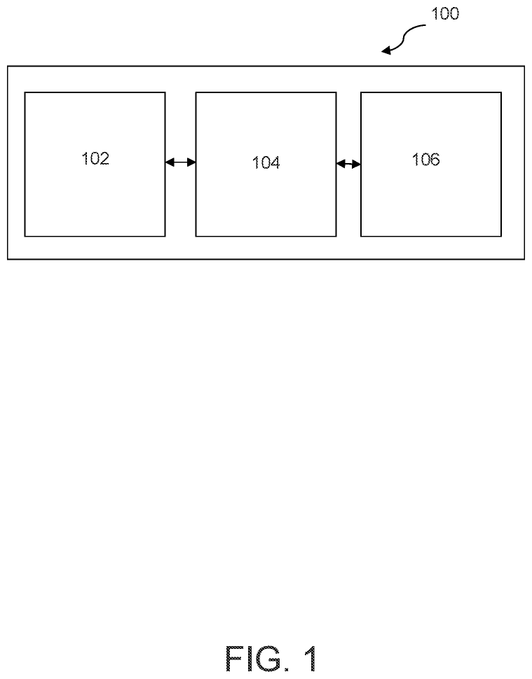

Referring to FIG. 1, there is shown a schematic illustration of a block diagram of a system 100 of prioritizing a plurality of secondary target protein in accordance with an embodiment of the present disclosure. The system 100 comprises a database arrangement 102 and a processor 106 communicably coupled via a data communication network 104 to the database arrangement 104.

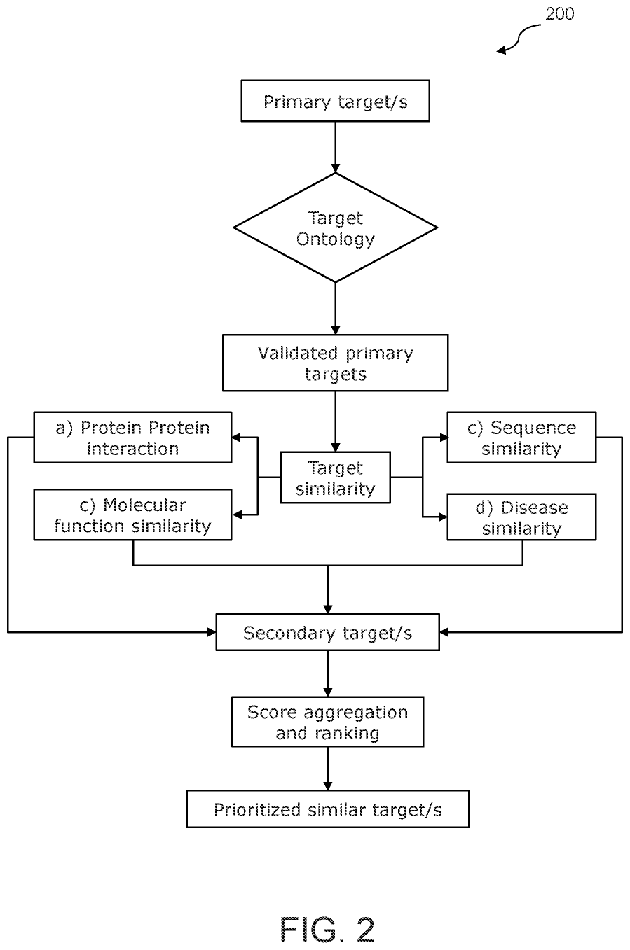

Referring to FIG. 2, there is shown a schematic illustration of a flow diagram 200 of the system for determining and prioritizing a plurality of secondary target protein, in accordance with an embodiment of the present disclosure. Herein, system comprises a database arrangement and a processor communicably coupled via a data communication network to the database arrangement. The processor is configured to receive information associated with a primary target protein. The processor further exercises step so as to utilize one or more ontologies in order to validate the received information associated with the one or more primary target protein. The validation of the primary target protein is based on the authentication and abstraction of data via one or more ontologies, wherein the data relates to the information associated with the one or more primary target protein. Further, the processor determines a plurality of secondary target protein similar to the primary target protein based on a set of similarity criteria, wherein the set of similarity criteria comprises at least protein-protein interaction, molecular function similarity, protein sequence similarity, and disease target similarity. Furthermore, each of the identified plurality of secondary target protein is being assigned a rank or chronological score, based on the matrix and graph analysis of each of the plurality of secondary target protein. The plurality of the secondary target protein is being prioritized and the steps for prioritization are shown below:
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

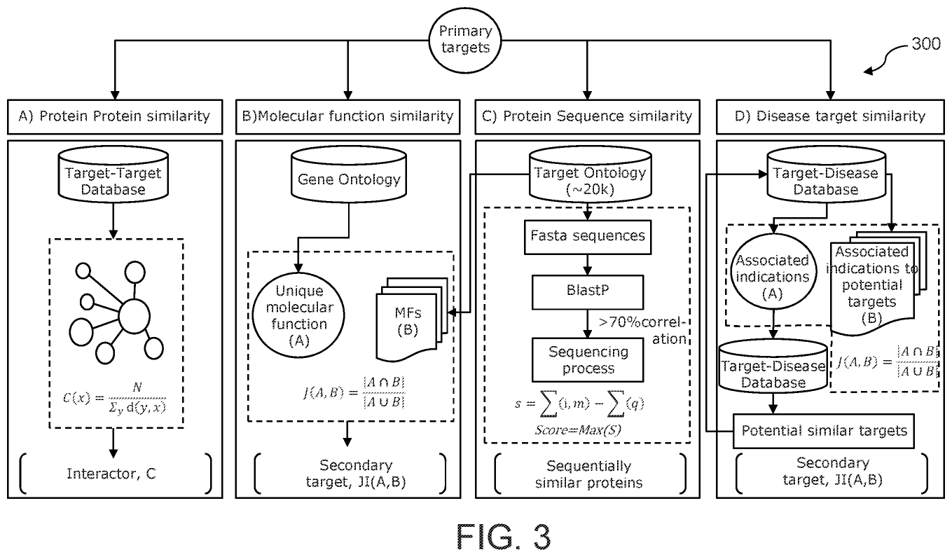

Referring to FIG. 3, there is shown a schematic illustration 300 of the determination of similar proteins as secondary target protein, by system 100 in accordance with an embodiment of the present disclosure. Herein, the processor receives the primary target protein, validates the received primary target protein using one or more ontologies and utilizes the validated primary target protein to determine the plurality of secondary target protein. The plurality of secondary target protein is identified similar to the primary target protein based on a set of similarity criteria, wherein the set of similarity criteria comprises at least one of protein-protein interaction, molecular function similarity, protein sequence similarity, and disease target similarity or their combination thereof. The protein-protein interaction is specific for different proteins and occurs between the defined binding regions of the proteins that facilitate having a particular biological function. In an embodiment, the protein-protein interaction is based on the closeness centrality of one or more primary target protein with respect to the plurality of secondary target protein. The closeness centrality is calculated as 1/(average of distance to all nodes in a network) where N is total number of nodes and d(y, x) denotes the distance between node(y) and node(x). On the other hand, the “molecular function” refers to the process that can be carried out by the action of a single macromolecular machine, usually via direct physical interactions with other molecular entities. Herein, the function denotes an action, or activity, that a gene product performs. These actions are described from two distinct but related perspectives: (1) biochemical activity, and (2) role as a component in a larger system/process. Optionally, the molecular function similarity score was calculated using the Jaccard index.
where,
A=(Molecular function target A),
B=(Molecular function target B)

$$J ( A , B ) = \frac{\vert A ⋂ B \vert}{\vert A ⋃ B \vert}$$

Furthermore, the protein sequence relates to the practical process of determining the amino acid sequence of all or part of a protein. This may serve to identify the protein or characterize its post-translational modifications. Herein, the post-translational modification (PTM) refers to the covalent and generally enzymatic modification of proteins following protein biosynthesis. Large-scale protein sequence comparison is an important but compute-intensive task in molecular biology. The another similarity criteria “disease target similarity” is another similarity criteria to be employed, relates to the target disease that a protein has its effect on for the disease treatment. Based on the assumption that similar proteins with similar functions tend to be associated with similar diseases, and vice versa, the interaction profile of protein p(i) is denoted by a binary vector IP(p(i)) representing whether protein p(i) is interacted with each disease or not. Then, the kernel for the two proteins p(i) and p(j) are defined to calculate the Gaussian kernel similarity based on their interaction profiles, which are defined as follows:

$$\begin{matrix}{KM}_{1} ( p ( i ) , p ( j ) ) = exp ( - {γ}_{p} {‖ IP ( p ( i ) ) - IP ( p ( j ) ) ‖}^{2} ) \\ {γ}_{p} = {γ}^{′} / ( \frac{1}{np} {\sum }_{γ = 1}^{np} {‖ IP ( p ( i ) ) ‖}^{2} )\end{matrix}$$

where γp is calculated by normalizing γ′p which divides the average number of associated diseases for all targets. γ′p, is set to 1 again.

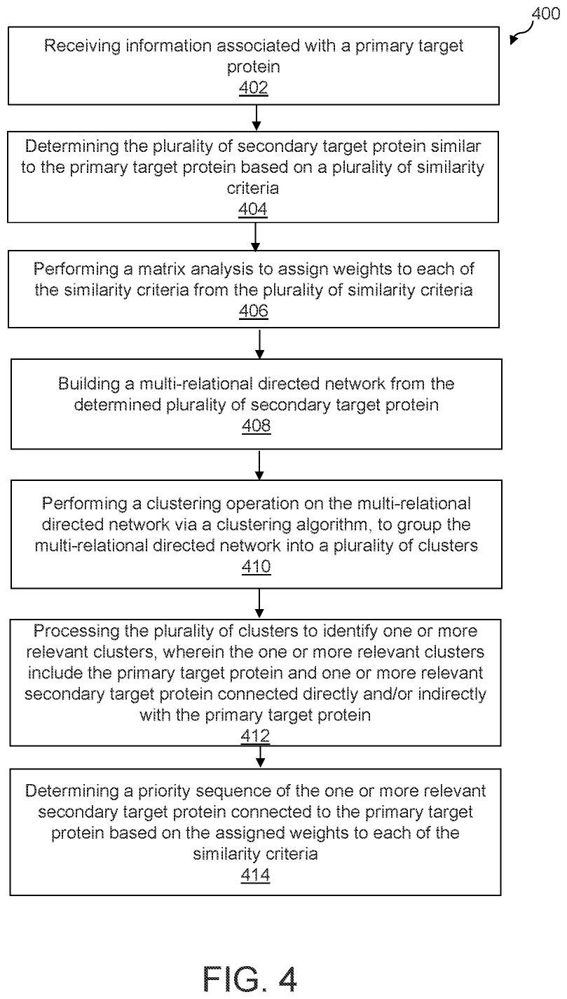

Referring to FIG. 4, there is shown a schematic illustration of the steps of method 400 of prioritizing a plurality of secondary target protein, in accordance with an embodiment of the present disclosure. At step 402, the method comprises receiving information associated with a primary target protein. At step 404, the method comprises determining the plurality of secondary target protein similar to the primary target protein based on a plurality of similarity criteria. Furthermore, in step 406, the method comprises performing a matrix analysis to assign weights to each of the similarity criteria from the plurality of similarity criteria. Thereafter at step 408, a multi-relational directed network is built from the determined plurality of secondary target protein by the processor. At step 410, the method comprises performing a clustering operation on the multi-relational directed network via a clustering algorithm, to group the multi-relational directed network into a plurality of clusters. At further step 412, the method comprises processing the plurality of clusters to identify one or more relevant clusters, wherein the one or more relevant clusters include the primary target protein and one or more relevant secondary target protein connected directly and/or indirectly with the primary target protein. At the final step 414, a priority sequence of the one or more relevant secondary target protein connected to the primary target protein is determined, based on the assigned weights to each of the similarity criteria

The steps 402, 404, 406, 408, 410, 412 and 414 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

1. A system for prioritizing a plurality of secondary target protein, wherein the system comprises:
a database arrangement; and
a processor communicably coupled via a data communication network to the database arrangement, wherein the processor is configured to:
receive information associated with a primary target protein;
determine the plurality of secondary target protein similar to the primary target protein based on a plurality of similarity criteria;
perform a matrix analysis to assign weights to each of the similarity criteria from the plurality of similarity criteria;
build a multi-relational directed network from the determined plurality of secondary target protein;
perform a clustering operation on the multi-relational directed network via a clustering algorithm, to group the multi-relational directed network into a plurality of clusters;
process the plurality of clusters to identify one or more relevant clusters, wherein the one or more relevant clusters include the primary target protein and one or more relevant secondary target protein connected directly and/or indirectly with the primary target protein; and
determine a priority sequence of the one or more relevant secondary target protein connected to the primary target protein based on the assigned weights to each of the similarity criteria.

2. The system of claim 1, wherein the processor is configured to validate the received information associated with the primary target protein based on authentication and abstraction of data using one or more ontologies, wherein the data relates to the information associated with the primary target protein.

3. The system of claim 2, wherein the one or more ontologies correspond to at least a protein ontology and a gene ontology.

4. The system of claim 1, wherein the set of similarity criteria comprises at least one of protein-protein interaction, molecular function similarity, in protein sequence similarity, and disease target similarity.

5. The system of claim 1, wherein the data that describes the primary target protein comprises at least sequence information, function classification information, metabolic pathway information, interaction profile, and Gene Ontology functional annotation of the primary target protein.

6. The system of claim 4, wherein the protein-protein interaction is based on closeness centrality of the primary target protein with respect to the plurality of secondary target protein.

7. The system of claim 1, wherein the processor is configured to assign weights to each of the similarity criteria based on an analysis of multi-criteria decision-making matrix.

8. The system of claim 7, wherein the processor is configured to calculate priority scores associated with each of the similarity criteria for calculating the weights to be assigned to each of the similarity criteria, wherein the priority scores determine the importance of a similarity criteria with respect to other similarity criteria.

9. The system of claim 1, wherein the multi-relational directed network defines a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

10. The system of claim 1, wherein the processor is configured to calculate direct scores and/or indirect scores of the relevant secondary target protein based on the weights of the similarity criteria assigned to each of the edges defined in the multi-relational directed network.

11. A computer-implemented method for prioritizing a plurality of secondary target protein, wherein the method comprises:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.

12. The computer-implemented method of claim 11, wherein the method comprises validating the received information associated with the primary target protein based on authentication and abstraction of data using one or more ontologies, wherein the data relates to the information associated with the primary target protein.

13. The system of claim 12, wherein the one or more ontologies correspond to at least a protein ontology and a gene ontology.

14. The system of claim 11, wherein the set of similarity criteria comprises at least one of protein-protein interaction, molecular function similarity, protein sequence similarity, and disease target similarity.

15. The system of claim 11, wherein the data that describes the primary target protein comprises at least sequence information, function classification information, metabolic pathway information, interaction profile, and Gene Ontology functional annotation of the primary target protein.

16. The system of claim 14, wherein the protein-protein interaction is based on closeness centrality of the primary target protein with respect to the plurality of secondary target protein.

17. The system of claim 11, wherein the method comprises assigning weights to each of the similarity criteria based on an analysis of multi-criteria decision-making matrix.

18. The system of claim 17, wherein the method comprises calculating priority scores associated with each of the similarity criteria for calculating the weights to be assigned to each of the similarity criteria, wherein the priority scores determine the importance of a similarity criteria with respect to other similarity criteria.

19. The system of claim 11, wherein the method comprises defining a plurality of nodes and one or more edges connected said plurality of nodes, wherein each of the nodes in the multi-relational directed network, corresponds to the plurality of secondary target proteins, further wherein the one or more edges in the multi-relational directed network, corresponds to each of the similarity criteria and the weights assigned therewith.

20. The system of claim 11, wherein the method comprises calculating direct scores and/or indirect scores of the relevant secondary target protein based on the weights of the similarity criteria assigned to each of the edges defined in the multi-relational directed network.

21. A non-transitory computer readable storage medium, containing program instructions for execution on a computer system, which when executed by a computer, cause the computer to perform method steps of a method for prioritizing a plurality of secondary target protein, the method comprising the steps of:
receiving information associated with a primary target protein;
determining the plurality of secondary target protein similar to the primary target protein based on a plurality of similarity criteria;
performing a matrix analysis to assign weights to each of the similarity criteria from the plurality of similarity criteria;
building a multi-relational directed network from the determined plurality of secondary target protein;
performing a clustering operation on the multi-relational directed network via a clustering algorithm, to group the multi-relational directed network into a plurality of clusters;
processing the plurality of clusters to identify one or more relevant clusters, wherein the one or more relevant clusters include the primary target protein and one or more relevant secondary target protein connected directly and/or indirectly with the primary target protein; and
determining a priority sequence of the one or more relevant secondary target protein connected to the primary target protein based on the assigned weights to each of the similarity criteria.
